(19) United States
(12) Patent Application Publication
(10) Pub. No.: US 2026/0295024 A1
(43) Pub. Date: Oct. 1, 2026

(54) KITS FOR TRICHINELLA VACCINATION BASED ON A PRIME-BOOST SEQUENTIAL IMMUNIZATION REGIMEN

(71) Applicant: JILIN UNIVERSITY, Changchun (CN)

(72) Inventors: Xiaolei LIU, Changchun (CN); Xue BAI, Changchun (CN); Zhina LIU, Changchun (CN); Mingyuan LIU, Changchun (CN); Nuo SU, Changchun (CN); Xuemin JIN, Changchun (CN); Ning XU, Changchun (CN); Bin TANG, Changchun (CN)

(73) Assignee: JILIN UNIVERSITY, Changchun (CN)

(21) Appl. No.: 19/679,577

(22) Filed: May 15, 2026

Related U.S. Application Data
(63) Continuation of application No. PCT/CN2025/143728, filed on Dec. 19, 2025.

(30) Foreign Application Priority Data
Dec. 24, 2024 (CN) 202411907957.4

(51) Int. Cl. A61K 39/00; A61P 37/04

(52) U.S. Cl. CPC A61K 39/0003; A61P 37/04; A61K 2039/5256

(57) ABSTRACT

Provided is a kit for a Trichinella vaccination, the kit including into two parts, a first part being a Trichinella recombinant protein vaccine that includes a Trichinella recombinant protein with an amino acid sequence as shown in SEQ ID NO:2, and a second part being a recombinant adenovirus expressing a protein with an amino acid sequence as shown in SEQ ID NO:1.

### CROSS-REFERENCE TO RELATED APPLICATIONS

This application is a continuation of International Application No. PCT/CN2025/143728, filed on Dec. 19, 2025, which claims priority to Chinese Application No. 202411907957.4, filed on Dec. 24, 2024, the entire contents of each of which are incorporated herein by reference.

### SEQUENCE LISTING

The instant application contains a Sequence Listing which has been submitted electronically in XML format and is hereby incorporated by reference in its entirety. The XML copy, created on Apr. 24, 2026, is named “2026-04-24-Sequence Listing-66706-H019US00” and is 6 bytes in size.

### TECHNICAL FIELD

The present disclosure generally relates to a field of genetic engineering technology, and in particular to a kit for a Trichinella vaccination based on a prime-boost sequential immunization regimen.

### BACKGROUND

Trichinellosis is a zoonotic parasitic disease caused by infection with Trichinella spiralis. The disease poses a huge threat to the livestock breeding industry, leading to hindered animal growth, reduced meat quality, and even animal death, resulting in direct economic losses. At the same time, humans may also contract the disease by consuming meat infected with Trichinella, leading to serious health problems, further affecting food safety and public health. Therefore, trichinellosis not only affects the sustainable development of the livestock breeding industry but also poses a potential risk to human health. Effective prevention and control measures are required to ensure the safety of livestock production and human health.

Although trichinellosis has caused a serious impact on the animal economy and the livestock breeding industry, there is currently no effective vaccine for trichinellosis. Traditional vaccine development methods, such as attenuated live vaccines and natural antigen vaccines, cannot meet the needs of antigens for immunodiagnosis and immunoprophylaxis because the antigenic components of Trichinella are complex, and Trichinella cannot complete its entire life cycle through in vitro culture, making it difficult to prepare such antigens through standardized mass production methods using artificially infected animals. With the rapid development of genetic engineering technology, novel vaccines such as recombinant protein vaccines, polypeptide vaccines, and DNA vaccines have been continuously explored and applied in the field of trichinellosis prevention and control. Although these vaccines possess a certain degree of protective efficacy, none of these vaccines have achieved complete protection against Trichinella infection.

### SUMMARY

The present disclosure provides a kit for a Trichinella vaccination. The kit includes two parts. A first part of the kit includes a Trichinella recombinant protein vaccine that includes a Trichinella recombinant protein with an amino acid sequence as shown in SEQ ID NO:2. A second part of the kit includes a recombinant adenovirus expressing a protein with an amino acid sequence as shown in SEQ ID NO:1.

### BRIEF DESCRIPTION OF THE DRAWINGS

FIG. 1 is a diagram of a recombinant adenovirus plasmid rAd5TsCLP and identification results, wherein M represents a DL15000 DNA Marker; lane 1 represents the recombinant adenovirus plasmid rAd5TsCLP; lane 2 represents identification results of PacI enzyme digestion linearization;

FIG. 2 is a diagram of results of indirect immunofluorescence assay (IFA) analysis of Ts-CLP protein expression after HEK293A cells are infected with a recombinant adenovirus rAd5TsCLP;

FIG. 3 is a diagram of results of signal peptide prediction for a Trichinella protein;

FIG. 4 is a flowchart of prokaryotic expression and immunization of a recombinant protein;

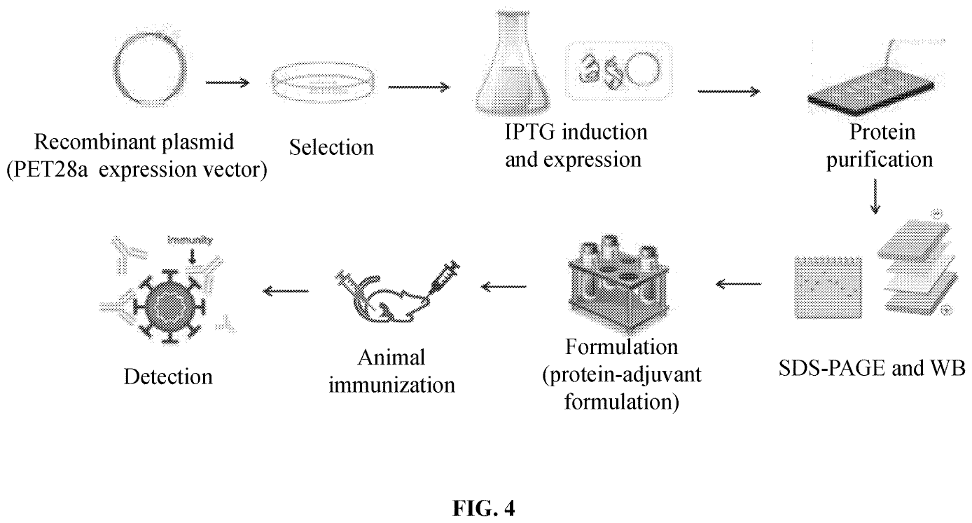

FIG. 5 is a diagram of results of sodium dodecyl sulfate-polyacrylamide gel electrophoresis (SDS-PAGE) and Western blot (WB) verification of Ts-CLP protein expression of a Trichinella recombinant protein vaccine;

FIG. 6 is a diagram of a Prime-boost animal immunization schedule and a diagram of specific immunization dose results; A represents the animal immunization schedule; B represents the immunization dose results;

FIG. 7 is a diagram of protective analysis results after mice immunized with rAd5TsCLP are challenged with 250 Trichinella muscle larvae, wherein A represents an average count of larvae per gram of muscle (LPG), B represents an average count of adult AD3 per gram of muscle, and data are mean±SEM;

FIG. 8 is a diagram of results of hematoxylin and eosin (HE) staining treatment on diaphragm tissue;

FIG. 9 is a diagram of detection results of specific antibody levels of IgG, IgG1, IgG2a, IgA, IgM, and neutralizing antibody (NAb) in mouse serum (*, P<0.05; **, P<0.01; ***, P<0.001); A is a diagram of detection results of specific IgG antibody levels in mouse serum; B is a diagram of detection results of specific IgG1 antibody levels in mouse serum; C is a diagram of detection results of specific IgG2a antibody levels in mouse serum; D is a diagram of detection results of specific IgA antibody levels in mouse serum; E is a diagram of detection results of specific IgM antibody levels in mouse serum; F is a diagram of detection results of NAb levels in mouse serum;

FIG. 10 is a diagram of cytokine levels in mouse serum (*, P<0.05; **, P<0.01; ***, P<0.001); A is a diagram of results of IFN-γ levels in mouse serum; B is a diagram of results of TNF-α levels in mouse serum; C is a diagram of results of IL-4 levels in mouse serum; D is a diagram of results of IL-10 levels in mouse serum;

FIG. 11 is a diagram of counts of CD3+CD4+, CD3+CD8+, and CD3+CD4+CD8+T lymphocytes (*, P<0.05; **, P<0.01; ***, P<0.001).

### DETAILED DESCRIPTION

The present disclosure provides a kit for Trichinella vaccination. The kit is divided into two parts. A first part is a Trichinella recombinant protein vaccine that includes a Trichinella recombinant protein with an amino acid sequence as shown in SEQ ID NO:2, and a second part is a recombinant adenovirus expressing a protein with an amino acid sequence as shown in SEQ ID NO: 1.

The Trichinella recombinant protein vaccine refers to a vaccine prepared by using genetic engineering techniques to introduce specific antigen genes from Trichinella, which can stimulate a protective immune response of a body, into an expression system (such as Escherichia coli, yeast, or mammalian cells) for large-scale in vitro expression, followed by extraction and purification of the antigen protein, and then formulation with an appropriate adjuvant. The Trichinella recombinant protein vaccine includes a Ts-CLP protein from which a signal peptide has been removed. The recombinant adenovirus refers to a viral vector constructed by using a naturally occurring adenovirus as a scaffold, removing its pathogenic genes or replication essential genes (typically the E1/E3 region) through genetic engineering approaches, and inserting exogenous target antigen genes into the genome thereof. The recombinant adenovirus can express exogenous antigens by using the machinery of a host cell after infecting the host cell, but cannot infinitely replicate within a host.

In some embodiments, an amount of the Trichinella recombinant protein is 30 to 90 μg; and content of the recombinant adenovirus expressing the protein with the amino acid sequence as shown in SEQ ID NO:1 is 10⁷ to 10⁹ plaque-forming units (PFU). In some embodiments, the amount of the Trichinella recombinant protein may be 30 μg, 35 μg, 40 μg, 45 μg, 50 μg, 55 μg, 60 μg, 65 μg, 70 μg, 75 μg, 80 μg, 85 μg, 90 μg, or any range composed of two values listed above, and is not limited to the enumerated values, wherein other unenumerated values within the range are also applicable. In some embodiments, the content of the recombinant adenovirus expressing the protein with the amino acid sequence as shown in SEQ ID NO:1 may be 10⁷ PFU, 10⁸ PFU, 10⁹ PFU, or any range composed of two values listed above, and is not limited to the enumerated values, wherein other unenumerated values within the range are also applicable. In some embodiments, the amount of the Trichinella recombinant protein is 60 μg; and the content of the recombinant adenovirus expressing the protein with the amino acid sequence as shown in SEQ ID NO: 1 is 10⁸ PFU. The immunization route of the Trichinella recombinant protein vaccine is intramuscular injection. The immunization route of the recombinant adenovirus expressing the protein with the amino acid sequence as shown in SEQ ID NO: 1 is intranasal administration.

In some embodiments, in the Trichinella recombinant protein vaccine, a volume ratio of the Trichinella recombinant protein with the amino acid sequence as shown in SEQ ID NO:2 to CPG1018 adjuvant is 3:1 to 1:2. In some embodiments, in the Trichinella recombinant protein vaccine, the volume ratio of the Trichinella recombinant protein with the amino acid sequence as shown in SEQ ID NO:2 to the CPG1018 adjuvant may be 3:1, 2:1, 1:1, 1:2, or any range composed of two values listed above, and is not limited to the enumerated values, wherein other unenumerated values within the range are also applicable. In some embodiments, in the Trichinella recombinant protein vaccine, the volume ratio of the Trichinella recombinant protein with the amino acid sequence as shown in SEQ ID NO:2 to the CPG1018 adjuvant is 1:1. The CPG1018 adjuvant is a synthetically produced short-chain DNA oligonucleotide and belongs to the class of Toll-like receptor 9 (TLR9) agonists. A core function of the CPG1018 adjuvant is to serve as a vaccine additive that significantly enhances and modulates an adaptive immune response elicited by the vaccine by activating specific innate immune pathways.

The present disclosure provides an application of the kit described above for use in preparing a medicament for preventing or treating trichinellosis. In some embodiments, the present disclosure provides a method for preventing or treating trichinellosis. The method includes administering the kit described above to an animal or a patient afflicted with trichinellosis.

The present disclosure provides a Trichinella recombinant protein, an amino acid sequence of which is as shown in SEQ ID NO:1 or SEQ ID NO:2.

The present disclosure provides a nucleic acid molecule of the Trichinella recombinant protein vaccine described above. The nucleic acid molecule of the Trichinella recombinant protein vaccine refers to an optimized codon nucleotide sequence suitable for expression in mammalian cells.

The present disclosure provides a recombinant vector of the nucleic acid molecule described above. The recombinant vector of the nucleic acid molecule refers to an artificial deoxyribonucleic acid (DNA) molecule in which a target nucleic acid molecule is spliced together with a DNA molecule (such as a plasmid or a viral genome backbone) that can autonomously replicate within a host cell.

The present disclosure provides a recombinant microbial cell including the nucleic acid molecule described above. The recombinant microbial cell including the nucleic acid molecule described above refers to an engineered unicellular organism (typically Escherichia coli, yeast, or the like) into which the recombinant vector is introduced through transformation or transfection techniques, and which is capable of stably maintaining, replicating the vector, or controllably expressing a target protein.

The present disclosure provides an application of the Trichinella recombinant protein described above, the nucleic acid molecule described above, the recombinant vector described above, or the recombinant microbial cell described above in preparing the vaccine or the medicament for preventing or treating trichinellosis.

In some embodiments, the vaccine is in the form of a nasal spray, an oral preparation, a suppository, or a parenteral preparation.

### Example 1. Preparation of Recombinant Adenovirus Vaccine (Also Referred to as Recombinant Adenovirus)

1. Construction of the rAd5TsCLP Recombinant Adenovirus

A Ts-CLP target gene was obtained. The Coding Sequence (CDS) (SEQ ID NO:3) of Ts-CLP was obtained from PubMed. An optimized codon nucleotide sequence suitable for expression in mammalian cells was designed, with a total length of 1218 base pairs (bp). The Ts-CLP gene fragment was amplified and identified. A recombinant adenovirus expressing the Ts-CLP gene was constructed using an AdEasy system of type 5 adenovirus with E1 and E3 deletions. A pShuttle-CMV-Ts-CLP shuttle plasmid was constructed. It was transformed into DH5a competent bacteria for amplification. After PmeI enzyme digestion, the linearized pShuttle-CMV-Ts-CLP shuttle plasmid was obtained. It was transformed into BJ5183 bacteria already carrying a pAdEasy-1 plasmid for homologous recombination. Finally, selection was performed using kanamycin.

SEQ ID NO: 3:
ATGAGCTTCATGCACTGCATCTTCGTGGTGCTCTTCTTCGCCGTGGGGG
AGGCCCAGATCCTCGGGGAGACAACCCACTACGGGAGGAACGACCCCGT
GATGCTGAGGAACGCCCACGAGGCCCTGTTCAGCAGCGATCTGAAGCAG
GAGAGCGGGGTGTTCCACAAGCTGCTGGAGCTGGAGGAGTCCAGCACCA
TGGGCATCCTGACAACCATGAAGGTCGTGATGCAGGACACCGACTGCCC
CGTGAGCTTCGCCCTGCTGAGCTACTACGATGTCCTGGTGAACTGCCAG
GGCGAGGGGAGGAGGAAGCACTGCACCATGGAGTACACACACCGGAACC
CCAGCAAGGCCACAGTGAGCAAGTGCTTCGAGGAGGTGGAGGAGCCCCT
GATCATCCCCCAGAGGGTGAAGATGATCGGCGGCCGGGCCGTGTACATC
GATAGCAACGCCGATGTGGAGGAGCAGATGCAGATGCTGGGCGAGACAA
CCCACTACGGCCGGAACGATCCCGTGATGCTGCCCAAGGCCAGGGAGGC
CCTCTTCAGCAGCGATAGCAAGGAGCAGAGCGGGGTCCTGCACAAGCTG
GTGGAGCTGGAGGAGTCCTCCACCATGGGGATTCTGACTACCATGAAGG
TCGTGATCCAGGACACCGAGTGCCGGGTGAGCAGCGCCTACAGCAGCTA
CTACGACGTGCTCCACTACTGCCACGGGAAGGGCCCCCGGAAGCACTGC
ACACTGGAGTACCGGCACCGGACACCCAGCACTGCCACAGTGTCCGAGT
GCTTCGAGGAGGTGGAGGAGCCACTGATCGTGCCCCAGAGGGTGCAGCG
GGTGAACGGGCGGACAATCTACCTGGACTCCAGCGATGACGTGGAGGAG
CAGGTGGTGTCCCAGAGGAGCCAGATGCTGGGCGGGACAACAAAGTACA
CCGACAGCAACGTGCACATCAAGGAGGAGGTGAAGCAGGCCATCTTCGA
GAGCGATAAGAAGAAGTCCTCCGGCACCTACCTGCTGCTGGATAAGATT
GTCGAGGGCTTTAACATGGGCATCAGCAGCCGGTTCCAGGTGCTGGTGA
AGGAGACCGAGTGCGGGATCAAGGAGAAGGCCTTCAACTCCTACGAGGA
CGTGTACAAGAACTGCAGCGGGTCCGGGGATAGCAAGGTGTGCTCCGTG
GAGTACAAGTACTTCGACCCCACCAAGAGCACCGTGGAGTGC.

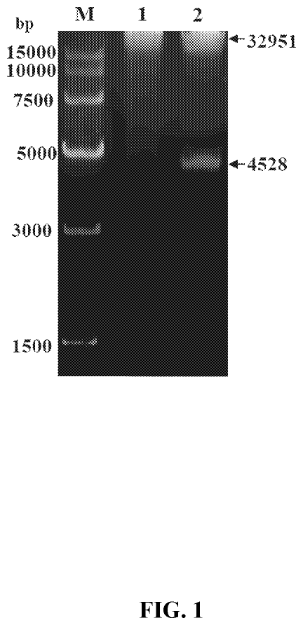

A small amount of the amplified BJ5183 bacteria was used to extract plasmids, and then identified by PacI enzyme digestion. After PacI enzyme digestion, the recombinant adenovirus plasmid yielded a large fragment of about 30 Kilobases (Kb) and a small fragment of 3 Kb or 4.5 Kb. The correctly identified rAd5TsCLP recombinant adenovirus plasmid was transformed into XL10-Gold supercompetent bacteria for amplification. The linearized recombinant adenovirus plasmid obtained after PacI enzyme digestion was used for adenovirus packaging after DNA purification (FIG. 1).

2. Packaging of the rAd5TsCLP Recombinant Adenovirus

Human Embryonic Kidney 293A (HEK293A) cells were inoculated at 7-8×10⁵ cells/mL into a 60-mm cell culture dish and cultured for 24 hours. The PacI enzyme-digested recombinant adenovirus plasmid was transfected into the overnight-cultured HEK293A cells via Lipofectamine 3000. After the 6 hours of transfection, the cell culture medium was replaced with fresh cell culture medium, and culturing was continued for 7 to 10 days. The cells clearly showed a floating state. The adenovirus was collected to obtain the rAd5TsCLP recombinant adenovirus vaccine.

3. Collection and Amplification of the rAd5 TsCLP Recombinant Adenovirus

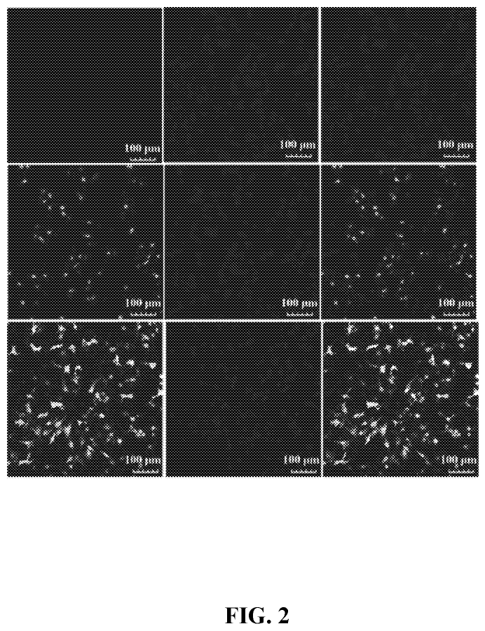

The cell culture medium was aspirated. 0.5 mL of Phosphate-Buffered Saline (PBS) was added, and the cells were scraped off and transferred to a centrifuge tube. The cells were lysed by repeated freeze-thaw cycles four times at −80° C. and 37° C. After centrifugation, the supernatant was collected to obtain a Passage 0 (P0) generation adenovirus, and saved for later use. HEK293A cells were infected with the P0 generation adenovirus for amplification. After a small amount of amplification and repeated freeze-thawing, a Passage 1 (P1) generation adenovirus was obtained. This was continued for three generations up to Passage 4 (P4) generation for large-scale amplification, purification, and concentration. After determining a titer, the rAd5 TsCLP recombinant adenovirus was aliquoted and stored. The results showed that IFA confirmed that rAd5TsCLP expressed the Ts-CLP protein in HEK293A cells (FIG. 2).

SEQ ID NO: 1:
MSFMHCIFVVLFFAVGEAQILGETTHYGRNDPVMLRNAHEALFSSDLKQ
ESGVFHKLLELEESSTMGILTTMKVVMQDTDCPVSFALLSYYDVLVNCQ
GEGRRKHCTMEYTHRNPSKATVSKCFEEVEEPLIIPQRVKMIGGRAVYI
DSNADVEEQMQMLGETTHYGRNDPVMLPKAREALFSSDSKEQSGVLHKL
VELEESSTMGILTTMKVVIQDTECRVSSAYSSYYDVLHYCHGKGPRKHC
TLEYRHRTPSTATVSECFEEVEEPLIVPQRVQRVNGRTIYLDSSDDVEE
QVVSQRSQMLGGTTKYTDSNVHIKEEVKQAIFESDKKKSSGTYLLLDKI
VEGFNMGISSRFQVLVKETECGIKEKAFNSYEDVYKNCSGSGDSKVCSV
EYKYFDPTKSTVEC.

### Example 2. Preparation of the Recombinant Protein Vaccine

### 1. Preparation of the Expression Bacteria.

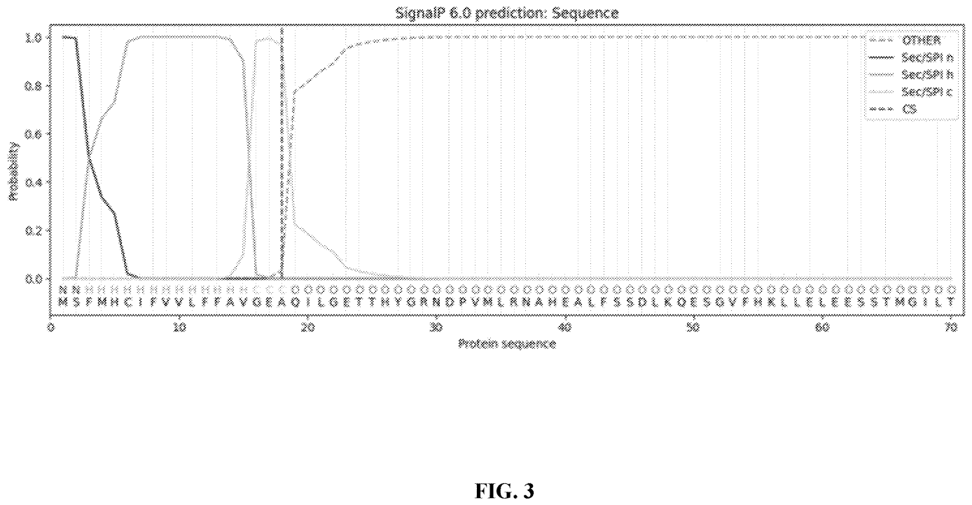

The SignalP 6.0 software was used to perform signal peptide prediction analysis on the sequence. A prokaryotic expression system was used to remove the signal peptide (amino acid sequence as shown in SEQ ID NO:2 after removing). The advantage is that it can avoid protein insolubility, inclusion body formation, and potential processing problems caused by the signal peptide, thereby improving the soluble expression and purification efficiency of the protein (FIG. 3).

SEQ ID NO: 2:
QILGETTHYGRNDPVMLRNAHEALFSSDLKQESGVFHKLLELEESSTMG
ILTTMKVVMQDTDCPVSFALLSYYDVLVNCQGEGRRKHCTMEYTHRNPS
KATVSKCFEEVEEPLIIPQRVKMIGGRAVYIDSNADVEEQMQMLGETTH
YGRNDPVMLPKAREALFSSDSKEQSGVLHKLVELEESSTMGILTTMKVV
IQDTECRVSSAYSSYYDVLHYCHGKGPRKHCTLEYRHRTPSTATVSECF
EEVEEPLIVPQRVQRVNGRTIYLDSSDDVEEQVVSQRSQMLGGTTKYTD
SNVHIKEEVKQAIFESDKKKSSGTYLLLDKIVEGFNMGISSRFQVLVKE
TECGIKEKAFNSYEDVYKNCSGSGDSKVCSVEYKYFDPTKSTVEC.

The Trichinella protein gene without the signal peptide was cloned into a pET-28a (+) vector and transformed into BL21 competent cells. After overnight culturing, a single clone was picked and cultured in Luria-Bertani (LB) medium for expansion to prepare pET28a-TsCLP expression bacteria, which were then stored at −80° C.

### 2. Induced Expression of the Recombinant Protein.

The pET28a-TsCLP expression bacteria stored in a −80° C. freezer were taken out. A loop was used to streak them on an LB plate containing kanamycin. A single colony was picked from the LB plate and inoculated into 5 mL of LB liquid medium, followed by expansion in 1 L of liquid LB medium. When the bacterial solution's Optical Density at 600 nm (OD600) was determined to be 0.4-0.6 by an enzyme-linked immunosorbent assay reader, 1 mL of 1 mol/L isopropyl-β-D-thiogalactoside (IPTG) inducer was added, and then cultured on a horizontal shaker at 37° C. for 6 hours. The induced bacterial solution was aliquoted into 500 mL centrifuge bottles. After centrifugation at 5000 g for 30 min, the supernatant was discarded, and the bacterial precipitate was collected. The bacterial precipitate was resuspended in PBS. The resuspended bacterial precipitate was added to 50 mL centrifuge tubes and subjected to three freeze-thaw cycles. The bacteria were resuspended and treated with an ultrasonic disruptor. The conditions for ultrasonic disruption were as follows: ultrasonic power of 200 W, duration of 3 s, interval of 3 s, total time of 45 min, and temperature of 4° C. The ultrasonically disrupted bacterial solution was centrifuged at 5000 g for 30 min. The supernatant was discarded, and the bacterial precipitate was collected (which contained the Ts-CLP protein, and after purification of the bacterial precipitate, a purified recombinant protein sample was obtained).

### 3. Gel Cutting and Purification.

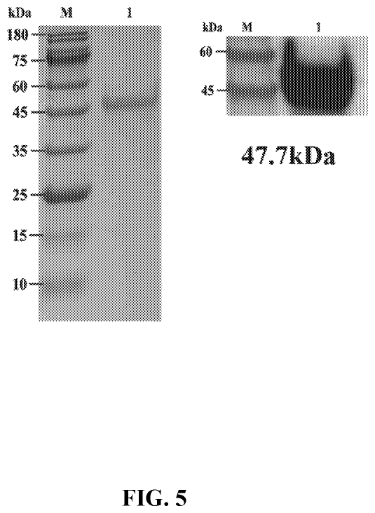

The purified recombinant protein sample was uniformly mixed with protein loading buffer according to a ratio. After boiling, it was used for SDS-PAGE electrophoresis. After the electrophoresis, the stacking gel was removed. The separating gel was placed in a 2.5% KCl solution and then placed in the 4° C. refrigerator. After 5 min, a white band appeared on the separating gel, and the white band was cut off with a sterile knife. The white band was cut into pieces and placed in a 2 mL centrifuge tube. An appropriate amount of autoclaved PBS solution was added, and it was shaken overnight in the 4° C. refrigerator. After centrifugation, the supernatant was drawn with a pipette gun. Then, the target protein was concentrated by centrifugation using a sterile ultrafiltration tube to a desired concentration. A large-volume endotoxin removal kit was used to remove endotoxins from the purified protein to obtain the recombinant Ts-CLP protein (rTsCLP). The Ts-CLP protein was verified by SDS-PAGE and Western blot (FIG. 5).

The rTsCLP recombinant protein vaccine (also referred to as recombinant protein vaccine rTsCLP) was prepared by mixing an adjuvant and the Trichinella recombinant protein with an amino acid sequence as shown in SEQ ID NO:2 at a 1:1 (v/v) volume ratio. Immunization was then performed.

### Example 3 Kit for Prime-Boost Immunization Vaccination

1. Kit Composition: A first part is the recombinant protein vaccine rTsCLP obtained in Example 2; a second part is the recombinant adenovirus rAd5TsCLP obtained in Example 1.
2. Specific Protocol for Strategic Animal Experiment

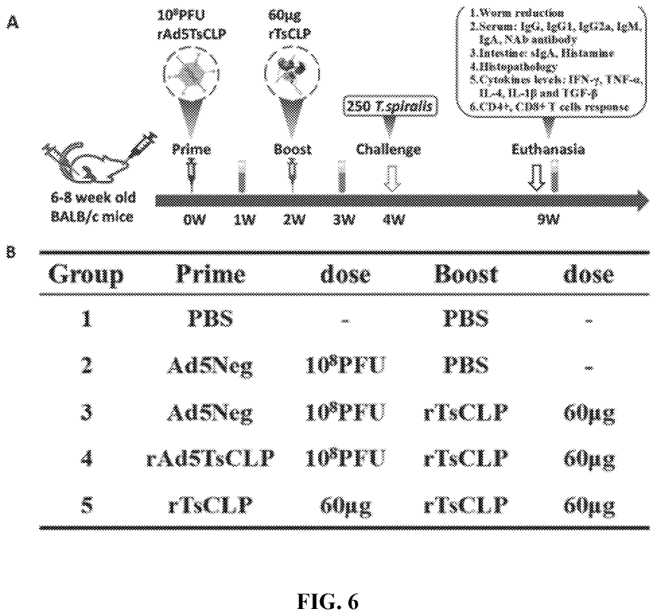

Six to eight-week-old female BALB/c mice were randomly divided into five groups, with 10 mice in each group. In week 0, 10⁸ PFU of recombinant adenovirus rAd5TsCLP was inoculated by intranasal administration. In week 2, 60 μg of the recombinant protein rTsCLP (rTsCLP to CPG1018 at a volume ratio of 1:1) was immunized via intramuscular injection into bilateral thighs. The total immunization volume was 100 μL (rAd5TsCLP and rTsCLP combined). Meanwhile, two immunizations with the recombinant protein vaccine (rTsCLP:rTsCLP) and a primary immunization with the adenovirus empty vector (also referred to as empty adenovirus Ad5Neg) followed by a boost immunization with the recombinant protein vaccine (Ad5Neg:rTsCLP) were used as vaccine control groups. At each immunization time point, an equivalent amount of PBS was administered as a blank control (also referred to as PBS control group), and 10⁸ PFU of an empty adenovirus Ad5Neg without the target gene was administered as an empty vector control. Two weeks after the last immunization, each mouse was orally infected with 250 Trichinella muscle larvae. On the 3rd day and 5th week after the challenge infection, the mice were euthanized, and a count of intestinal adult worms and muscle larvae were determined. Based on the collected count of muscle larvae and a muscle weight, the count of muscle larvae per gram of muscle (LPG) was determined. By comparison with the PBS control group, an average LPG of mice in each group was determined, and the reduction rate of larvae was determined. In weeks 1, 3, and 9, serum, spleen, and small intestine were collected, and the levels of humoral immunity, cellular immunity, and mucosal immunity in each group were detected. At the same time, the diaphragms of the mice were subjected to HE staining treatment for parasite load assessment (FIG. 6).

### Example 4 Evaluation of Immunization Efficacy of Prime-Boost Immunization Regimen

10⁸ PFU of the recombinant adenovirus rAd5TsCLP was inoculated into 6-8 week old female BALB/c mice (10 mice per group) respectively by intramuscular injection and intranasal administration. At the same time, a treatment involving inoculation with the adenovirus Ad5Neg was set as a vaccine control, and a treatment involving inoculation with PBS was set as a blank control.

Detection of Intestinal Adult Worm and Muscle Larvae in Mice: On the 3rd day and 5th week after Trichinella infection, the mice were euthanized, a count of intestinal adult worms and muscle larvae in the mice were determined, and a reduction rate of larvae was determined. The count of muscle larvae in the mice was determined by a pepsin digestion manner. The specific manner is as follows: pepsin and hydrochloric acid were added into distilled water at 37° C. to prepare the digestion solution. The final concentrations of the pepsin and the hydrochloric acid were both 1%. Each mouse was digested with 500 mL of the digestion solution on a magnetic stirrer in a 37° C. constant temperature incubator for 2 hours. After the entire digestion process, the residue in the digestion solution was filtered out with a 60-mesh sieve, and then allowed to stand for 1 hour at room temperature. The upper layer liquid was gently drawn out with a sterile syringe, and 100 mL of the liquid was retained. After re-adding 400 mL of distilled water and repeated washing, the liquid was finally transferred to a 50 mL beaker, and the Trichinella muscle larvae were counted under a microscope.

### 1. Determination of Reduction Rate of Muscle Larvae

Pepsin and hydrochloric acid at a final concentration of 1% were added to distilled water at 37° C. to prepare a 500 mL digestion solution for digesting each mouse for 2 hours. After the digestion, the residue was filtered with a 60-mesh sieve, allowed to stand for 1 hour, the upper layer liquid was drawn out and 100 mL was retained. After repeated washing, the liquid was transferred to a 50 mL beaker. Trichinella muscle larvae were counted under a microscope, and the count of muscle larvae per gram of muscle (LPG) in each mouse was determined. By comparison with the PBS control group, the reduction rate of larvae for each experimental group was determined, and the formula is as follows:

$$Reduction Rate of Muscle Larvae ( \% ) = 1 - ( the average LPG of the experimental \text{group/} LPG of the PBS control group ) \times 100 \%$$

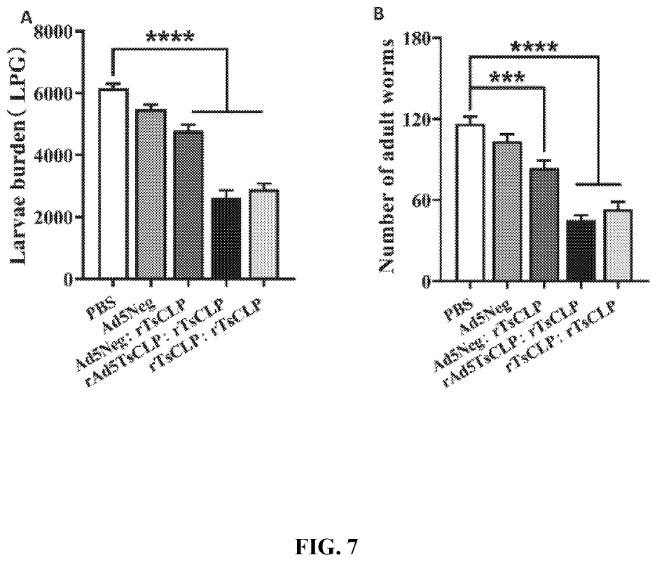

The results showed that five weeks after the challenge infection, compared with the PBS control group, the muscle larvae load decreased by 9.88%, 21.29%, 58.22%, and 53.39% respectively, and the adult worm load decreased by 9.28%, 34.36%, 61.17%, and 54.3% respectively for the Ad5Neg (adenovirus empty vector) group, the Ad5Neg:rTsCLP (primary immunization with adenovirus empty vector, boost immunization with recombinant protein) group, the rAd5TsCLP:rTsCLP (primary immunization with adenovirus, boost immunization with recombinant protein) group, and the rTsCLP:rTsCLP (two immunizations with recombinant protein) group (FIG. 7).

Combined immunization with rAd5TsCLP:rTsCLP reduced muscle larvae by 58.22%. Single immunization with rAd5TsCLP reduced muscle larvae by 45.61% via intramuscular injection and by 47.91% via intranasal administration.

Combined immunization with rAd5TsCLP:rTsCLP reduced adult worms by 61.17%. Single immunization with rAd5 TsCLP reduced adult worms by 36.49% via intramuscular injection and by 42.57% via intranasal administration.

### 2. HE Staining of Diaphragm Tissue

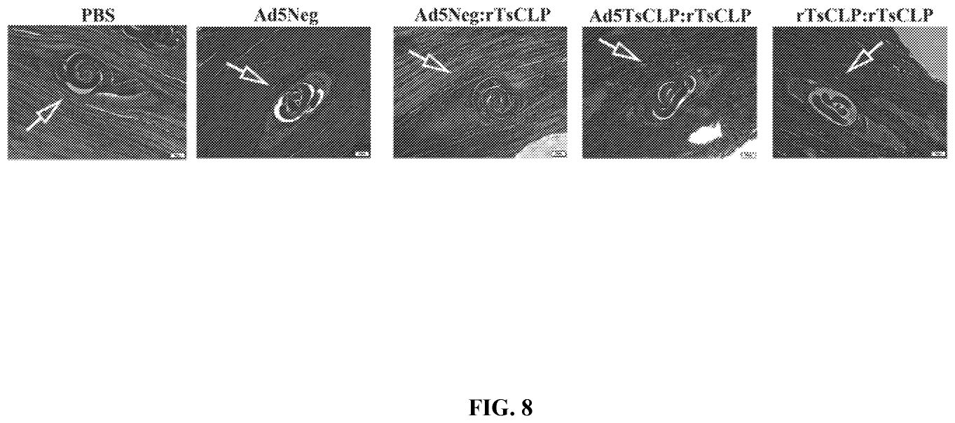

The diaphragm tissue was sectioned after steps such as dehydration, clearing, wax infiltration, embedding, or the like, and then subjected to HE staining to observe its tissue structure and pathological changes. The results showed that, compared with the PBS and Ad5Neg groups, more severe inflammatory cell infiltration occurred around the rAd5TsCLP:rTsCLP cysts, and the inflammatory cells entered inside the cysts to directly contact the worm body (FIG. 8).

### 3. Specific Antibody Levels

### Specific Antibody ELISA Detection

The specific antibodies (immunoglobulin G (IgG), immunoglobulin G1 (IgG1), immunoglobulin G2a (IgG2a), immunoglobulin M (IgM), and immunoglobulin A (IgA)) in the serum of mice in each group were determined using an indirect Enzyme-Linked Immunosorbent Assay (ELISA). 5 μg/mL of the purified recombinant protein rTsCLP was coated onto a 96-well plate and incubated overnight at 4° C. Subsequently, 100 μL/well of a blocking solution (5% skim milk) was added, and blocked for 1 hour at 37° C. The collected serum was subjected to 10-fold serial dilution. After incubation for 1 hour at 37° C., goat anti-mouse IgG-horseradish peroxidase (HRP) was added for incubation. Finally, a tetramethylbenzidine (TMB) substrate was added for color development, the reaction was stopped with 2M sulfuric acid (H₂SO₄), and the optical density at 450 nm (OD450) value was determined using a microplate reader.

### 4. Determination of Neutralizing Antibody NAb

The collected serum was inactivated at 56° C. for 30 minutes, and then subjected to a 2-fold serial dilution (1:10, 1:20, 1:40, 1:80, 1:160, 1:320, 1:640, and 1:1280 in 100 μL Dulbecco's Modified Eagle Medium (DMEM)) in a 96-well plate. The diluted serum was mixed with an equal volume of 100 Tissue Culture Infectious Dose (TCID₅₀) of the recombinant adenovirus rAd5TsCLP and incubated for 1 hour at 37° C. Subsequently, 200 μL of the serum-virus mixture was added to the monolayer HEK293A cells. Ad5Neg and HEK293A were used as control groups. After incubation for 72 hours under 37° C., 5% carbon dioxide (CO₂) conditions, the expression of a viral fluorescent protein was observed, and a neutralizing antibody titer was determined. If a count of green fluorescent foci in the sample dilution were reduced by more than 50% compared to the infected control group, the neutralizing antibody was determined to be positive.

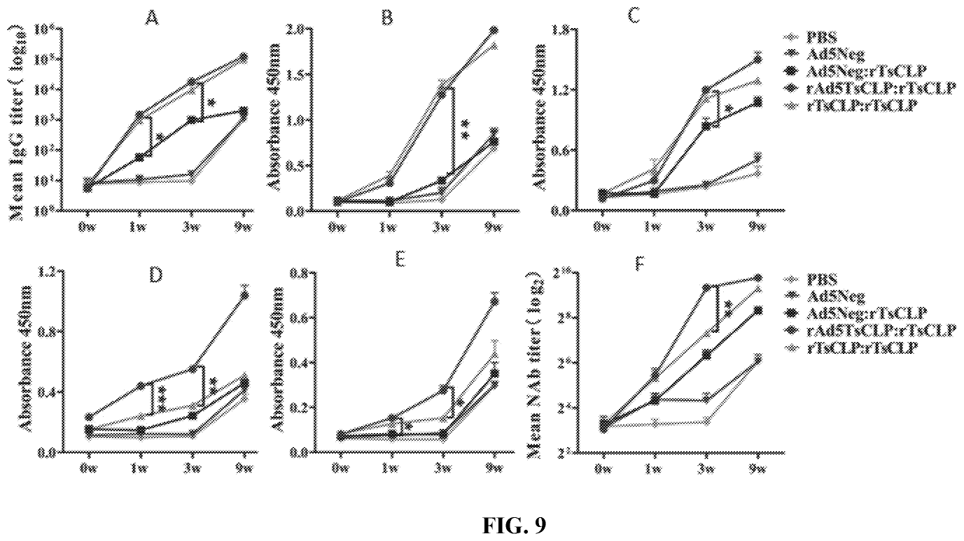

The results showed that rAd5TsCLP:rTsCLP can stimulate higher levels of IgG, IgG1, IgG2a, IgA, IgM, and NAb. After the challenge, throughout the entire process of the worm body from invading a host to completing its life cycle, these detected antibody levels still maintained a gradually increasing trend. This may mean that the host immune system continuously strengthens its defensive ability against worm invasion, attempting to resist potential harm that may be brought by the worm (FIG. 9).

Values of average NAb titers: the average antibody titer of the PBS group was 40 in week 7 (35 days post-challenge). The average antibody titer of the Ad5Neg group was 40 in week 7 (35 days post-challenge). The average antibody titer of the Ad5Neg:rTsCLP group was 320 in week 7 (35 days post-challenge). The average antibody titer of the rTsCLP:rTsCLP group was 640 in week 7 (35 days post-challenge). The average antibody titer of the rAd5 TsCLP:rTsCLP group was 1024 in week 7 (35 days post-challenge) (determined by a 2-fold serial dilution re-test). The average antibody titer of the rAd5 TsCLP group with single intramuscular injection inoculation was 512 in week 7 (35 days post-challenge) (determined by a 2-fold serial dilution re-test). The average antibody titer of the rAd5TsCLP group with single intranasal injection inoculation was 320 in week 7 (35 days post-challenge).

### 5. Serum Cytokine Detection

A secretion content of interferon-gamma (IFN-γ), tumor necrosis factor-alpha (TNF-α), interleukin-4 (IL-4), and interleukin-10 (IL-10) cytokines in the mouse serum was determined using a Luminex detection platform.

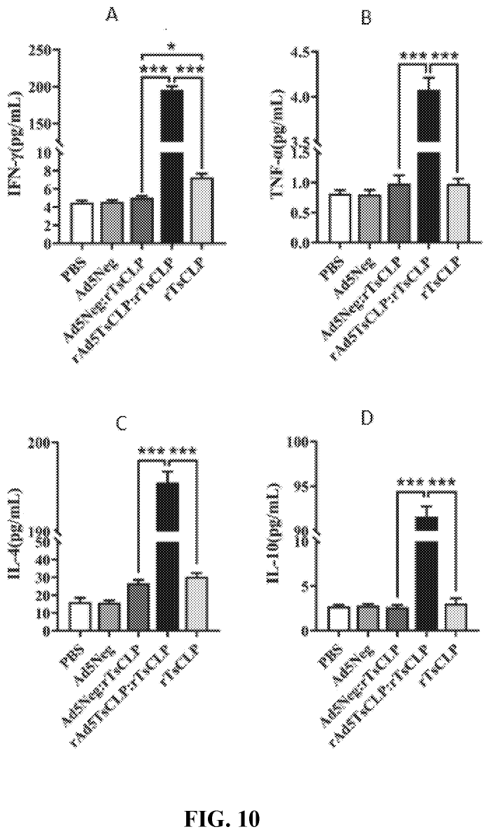

The results showed that, compared to other immunization groups, the concentrations of IFN-γ, TNF-α, IL-4, and IL-10 cytokines in the mouse serum of the rAd5 TsCLP:rTsCLP group were the highest. Although a t helper 2 (Th2) response and mucosal immunity play an important role in Trichinella clearance, a t helper 1 (Th1) response also plays an equally important role therein (FIG. 10).

### 6. Determination of T Lymphocytes

After the mice were euthanized, the mouse spleen was removed and separated into a single cell suspension, and then passed through a 100-mesh cell sieve. After lysis with a red blood cell lysis solution, the cells were resuspended in PBS. In the dark, the cells were stained with anti-mouse cluster of differentiation (CD) 3-allophycocyanin (APC), CD4-fluorescein isothiocyanate (FITC), and CD8-phycoerythrin (PE) surface marker antibodies for 30 minutes at 4° C. All samples were analyzed using a BD FACSCanton flow cytometer, and data analysis was performed using FlowJo 10 software.

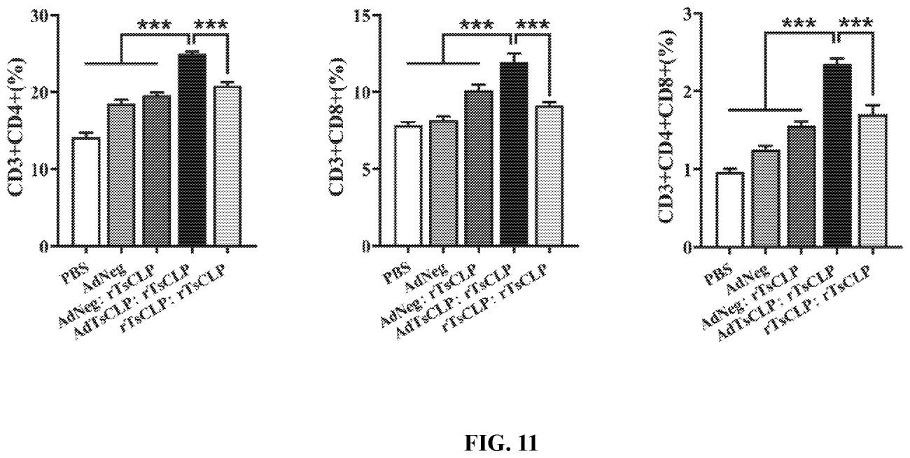

The results showed that, compared to the PBS group, the counts of CD3⁺CD4⁺ T, CD3⁺CD8⁺ T, and CD3⁺CD4⁺CD8⁺ T lymphocytes increased in other immunization groups, with the rAd5TsCLP:rTsCLP group showing the highest increase in the count of secreted T lymphocytes. Therefore, the prime-boost immunization regimen can stimulate T lymphocyte proliferation capability, promote the secretion of cytokines, and thereby regulate and enhance the strength and breadth of the immune response (FIG. 11).

The above results indicate that: (1) the present disclosure provides a method for preparing the recombinant adenovirus vaccine; (2) the present disclosure provides a method for preparing the Trichinella recombinant protein vaccine; (3) the present disclosure integrates advantages of the recombinant adenovirus vaccine and the Trichinella recombinant protein vaccine, and adopts the prime-boost immunization regimen to immunize BALB/c mice; (4) the prime-boost immunization regimen adopted in the present disclosure has a plurality of advantages, which may effectively enhance a systemic immunity level of the body, strongly stimulate the body to produce a strong mucosal immune response, promote Th1/Th2 to maintain a dynamic equilibrium state, and enhance a proliferation capability of T lymphocytes; and (5) the prime-boost immunization regimen described in the present disclosure exhibits unique advantages in coping with Trichinellosis. The prime-boost immunization regimen may effectively save immunization dosage, avoid a plurality of inconveniences and potential risks that may be brought by large-scale vaccination, and simultaneously significantly enhance immunization efficacy, such that the body may more powerfully resist the invasion of Trichinella, and provide new approaches and reliable ways for the prevention and control of Trichinellosis.

The present disclosure has the following beneficial effects: By using the recombinant adenovirus vaccine for primary immunization (prime) and the Trichinella recombinant protein vaccine for booster immunization (boost), the immunogenicity of the vaccine may be effectively enhanced, thereby stimulating the body to produce a stronger immune response. The regimen integrates high immunogenicity of the recombinant adenovirus vaccine and high safety of the Trichinella recombinant protein vaccine, synergistically enhances the immune response, and provides robust immune protection for the body. In addition, the prime-boost immunization regimen may also stimulate the body to form long-term immune memory. This means that once the body encounters Trichinella infection again, it can rapidly activate a strong immune response, rapidly clear the pathogen, thereby effectively reducing the risk of disease occurrence.

The prime-boost immunization regimen exhibits unique advantages in the development of Trichinella vaccinations, aiming to overcome limitations of a single vaccine type. The regimen first utilizes the recombinant adenovirus vaccine for primary immunization (prime), which can efficiently and rapidly activate humoral immunity and cellular immunity, including stimulating B lymphocytes to produce plasma cells that secrete antibodies, such as IgG, or the like, to neutralize viruses, and activating cytotoxic T lymphocytes to recognize and kill virus-infected target cells, thereby clearing viruses. However, homologous immunization with the recombinant adenovirus vaccine may lead to pre-existing immunity problems, reduce immunogenicity, and cause adverse reactions, such as fever, or the like. The Trichinella recombinant protein vaccine, as a booster (boost), may compensate for deficiencies of the recombinant adenovirus vaccine. It has strong immunogenicity, can significantly enhance NAb levels, and has high safety, few adverse reactions, good stability, and requires undemanding storage and transportation conditions. Therefore, the prime-boost immunization regimen not only effectively avoids deficiencies of single-type Trichinella vaccinations but also significantly enhances the efficacy and persistence of the immune response, and provides more efficient and comprehensive immune protection for the body.

Adenoviruses have the problem of pre-existing immunity. If both doses are vaccinated with the adenovirus vaccine, it will affect the immunization efficacy. The Trichinella recombinant protein vaccine requires adjuvants and 2-3 immunization doses. Considering that Trichinellosis vaccines are primarily applied to pigs (pigs are the main source of infection), and the market readiness of pigs has a time limit (approximately half a year). The adenovirus vaccine does not require adjuvants, and single-dose vaccination has high efficacy. Therefore, the adenovirus vaccine is used for primary immunization to activate the immune system, and only one dose of the Trichinella recombinant protein vaccine is used for booster immunization, thereby avoiding the problem of a long immunization cycle.

What is claimed is:

1. A kit for a Trichinella vaccination, comprising two parts, wherein a first part is a Trichinella recombinant protein vaccine that includes a Trichinella recombinant protein with an amino acid sequence as shown in SEQ ID NO:2, and a second part is a recombinant adenovirus expressing a protein with an amino acid sequence as shown in SEQ ID NO:1.

2. The kit according to claim 1, wherein an amount of the Trichinella recombinant protein is 60 μg; a content of the recombinant adenovirus expressing the protein with the amino acid sequence as shown in SEQ ID NO:1 is 10⁸ plaque-forming units (PFU); an immunization route of the Trichinella recombinant protein vaccine is intramuscular injection; and an immunization route of the recombinant adenovirus expressing the protein with the amino acid sequence as shown in SEQ ID NO: 1 is intranasal administration.

3. The kit according to claim 1, wherein in the Trichinella recombinant protein vaccine, a volume ratio of the Trichinella recombinant protein with the amino acid sequence as shown in SEQ ID NO:2 to a CPG1018 adjuvant is 1:1.


SEQUENCE LISTING (3 sequences)

SEQ ID NO: 1 - protein, 406 aa, Trichinella spiralis
MSFMHCIFVVLFFAVGEAQILGETTHYGRNDPVMLRNAHEALFSSDLKQESGVFHKLLELEESSTMGILTTMKVVMQDTDCPVSFALLSYYDVLVNCQGEGRRKHCTMEYTHRNPSKATVSKCFEEVEEPLIIPQRVKMIGGRAVYIDSNADVEEQMQMLGETTHYGRNDPVMLPKAREALFSSDSKEQSGVLHKLVELEESSTMGILTTMKVVIQDTECRVSSAYSSYYDVLHYCHGKGPRKHCTLEYRHRTPSTATVSECFEEVEEPLIVPQRVQRVNGRTIYLDSSDDVEEQVVSQRSQMLGGTTKYTDSNVHIKEEVKQAIFESDKKKSSGTYLLLDKIVEGFNMGISSRFQVLVKETECGIKEKAFNSYEDVYKNCSGSGDSKVCSVEYKYFDPTKSTVEC

SEQ ID NO: 2 - protein, 388 aa, Trichinella spiralis
QILGETTHYGRNDPVMLRNAHEALFSSDLKQESGVFHKLLELEESSTMGILTTMKVVMQDTDCPVSFALLSYYDVLVNCQGEGRRKHCTMEYTHRNPSKATVSKCFEEVEEPLIIPQRVKMIGGRAVYIDSNADVEEQMQMLGETTHYGRNDPVMLPKAREALFSSDSKEQSGVLHKLVELEESSTMGILTTMKVVIQDTECRVSSAYSSYYDVLHYCHGKGPRKHCTLEYRHRTPSTATVSECFEEVEEPLIVPQRVQRVNGRTIYLDSSDDVEEQVVSQRSQMLGGTTKYTDSNVHIKEEVKQAIFESDKKKSSGTYLLLDKIVEGFNMGISSRFQVLVKETECGIKEKAFNSYEDVYKNCSGSGDSKVCSVEYKYFDPTKSTVEC

SEQ ID NO: 3 - DNA, 1218 bp, Trichinella spiralis
  source 1..1218: mol_type = genomic DNA
atgagcttcatgcactgcatcttcgtggtgctcttcttcgccgtgggggaggcccagatcctcggggagacaacccactacgggaggaacgaccccgtgatgctgaggaacgcccacgaggccctgttcagcagcgatctgaagcaggagagcggggtgttccacaagctgctggagctggaggagtccagcaccatgggcatcctgacaaccatgaaggtcgtgatgcaggacaccgactgccccgtgagcttcgccctgctgagctactacgatgtcctggtgaactgccagggcgaggggaggaggaagcactgcaccatggagtacacacaccggaaccccagcaaggccacagtgagcaagtgcttcgaggaggtggaggagcccctgatcatcccccagagggtgaagatgatcggcggccgggccgtgtacatcgatagcaacgccgatgtggaggagcagatgcagatgctgggcgagacaacccactacggccggaacgatcccgtgatgctgcccaaggccagggaggccctcttcagcagcgatagcaaggagcagagcggggtcctgcacaagctggtggagctggaggagtcctccaccatggggattctgactaccatgaaggtcgtgatccaggacaccgagtgccgggtgagcagcgcctacagcagctactacgacgtgctccactactgccacgggaagggcccccggaagcactgcacactggagtaccggcaccggacacccagcactgccacagtgtccgagtgcttcgaggaggtggaggagccactgatcgtgccccagagggtgcagcgggtgaacgggcggacaatctacctggactccagcgatgacgtggaggagcaggtggtgtcccagaggagccagatgctgggcgggacaacaaagtacaccgacagcaacgtgcacatcaaggaggaggtgaagcaggccatcttcgagagcgataagaagaagtcctccggcacctacctgctgctggataagattgtcgagggctttaacatgggcatcagcagccggttccaggtgctggtgaaggagaccgagtgcgggatcaaggagaaggccttcaactcctacgaggacgtgtacaagaactgcagcgggtccggggatagcaaggtgtgctccgtggagtacaagtacttcgaccccaccaagagcaccgtggagtgc